(19) United States
(12) Patent Application Publication
(10) Pub. No.: US 2017/0319455 A1
(43) Pub. Date: Nov. 9, 2017

(54) METHOD FOR OCCLUDING DENTIN TUBULES AND REMINERALIZING TEETH

(71) Applicant: University of Dammam, Dammam (SA)

(72) Inventors: Imran FAROOQ, Dammam (SA); Imran A. MOHEET, Dammam (SA); Emad ALSHWAIMI, Dammam (SA)

(73) Assignee: University of Dammam, Dammam (SA)

(21) Appl. No.: 15/274,297

(22) Filed: Sep. 23, 2016

Related U.S. Application Data
(60) Provisional application No. 62/330,911, filed on May 3, 2016.

(51) Int. Cl. A61K 8/49; A61K 8/24; A61K 8/21; A61K 8/25; A61Q 11/00; A61K 8/64

(52) U.S. Cl. CPC A61K 8/4953; A61Q 11/00; A61K 8/64; A61K 8/21; A61K 8/25; A61K 8/24; A61K 2800/5922

(57) ABSTRACT

A method for occluding dentin tubules and remineralizing teeth with a toothpaste. The toothpaste may be applied onto a mouth tray and the teeth are contacted with the toothpaste present in the mouth tray wherein the toothpaste includes theobromine and at least one of a bioactive glass and hydroxyapatite.

### BACKGROUND OF THE INVENTION

### Field of the Invention

This disclosure relates to methods for occluding dentin tubules and remineralizing teeth.

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Dentin is a mineralized hard tissue containing abundant tiny dentin tubules (30,000-40,000/mm²) (Yadav K, Sofat A, Gambhir S R, Galhotra V. J Nat Sci Biol Med 2014, 5(1):21-4, incorporated herein by reference in its entirety). Dentin exposure resulting from loss of enamel and gingival recession is considered a key predisposing factor leading to dentin hypersensitivity (Addy M, West N. Curr Opin Periodontol 1994:71-7, incorporated herein by reference in its entirety). Dentin hypersensitivity is recognized as a “sharp pain of short duration which occurs when a stimulus of thermal, chemical, evaporative, tactile, or osmotic origin comes in contact with an exposed dentin surface” (Ali S, Farooq I. World J Dent 2013, 4(3):188-92, incorporated herein by reference in its entirety). Its prevalence varies between different populations, but generally, it is reported to be between 15 and 74% (Gillam D G. Clin Oral Investig 2013, 17(Suppl 1):21-9, incorporated herein by reference in its entirety). Although dentin hypersensitivity affects individuals of all age groups, many people suffering from dentin hypersensitivity have been reported to be between 30 and 40 years, with a majority being women (Miglani S, Aggarwal V, Ahuja B. J Conserv Dent 2010, 13(4):218-24; and Christian H S, Tachou A. Clin Oral Invest 2013, 17(Suppl. 1):S3-8, each incorporated herein by reference in their entirety). The most accepted theory explaining the mechanism of dentin hypersensitivity is the hydrodynamic theory. According to this theory, dentin hypersensitivity occurs because of the movement of fluid within dentin tubules in response to a stimulus acting on exposed dentin surfaces (Brännstróm M. Oral Surg Oral Med Oral Pathol 1966, 21(4):517-26, incorporated herein by reference in its entirety). A logical solution to reduce dentin hypersensitivity is to block the uncovered dentin tubules, which could result in decreased permeability and sensitivity (Gillam D G, Mordan N J, Newman H N. Adv Dent Res 1997, 11(4):487-501, incorporated herein by reference in its entirety).

In view of the foregoing, one objective of the present disclosure is to provide a method for occluding dentin tubules and remineralizing teeth.

### SUMMARY OF THE DISCLOSURE

The foregoing description is intended to provide a general introduction and summary of the present disclosure and is not intended to be limiting in its disclosure unless otherwise explicitly stated. The presently preferred embodiments, together with further advantages, will be best understood by reference to the following detailed description taken in conjunction with the accompanying drawings.

A first aspect of the disclosure relates to a method for occluding dentin tubules, the method comprising contacting the dentin tubules with a toothpaste for 1-5 minutes to form occluded dentin tubules which are resistant to an acid, wherein the toothpaste comprises theobromine and at least one of a bioactive glass and hydroxyapatite, a weight ratio of theobromine to the at least one of the bioactive glass and hydroxyapatite ranges from 5:95 to 95:5, the acid is citric acid or acetic acid, and at least 97% of the dentin tubules are occluded.

In one embodiment, the toothpaste is applied to a mouth tray and the dentin tubules are contacted with the toothpaste present in the mouth tray.

In one embodiment, the toothpaste comprises 10-30 wt % of theobromine, based on a total weight of the at least one of the bioactive glass and hydroxyapatite.

In one embodiment, the toothpaste comprises the bioactive glass and 5-95 wt % of hydroxyapatite, based on a weight of the bioactive glass.

In one embodiment, the toothpaste comprises hydroxyapatite, and further comprises 10-50 wt % of fluorapatite, based on a weight of hydroxyapatite.

In one embodiment, at least 99% of the dentin tubules are occluded.

In one embodiment, the bioactive glass is present and the bioactive glass comprises 40-80 wt % of silica and 20-50 wt % of calcium oxide, based on a weight of the bioactive glass.

In one embodiment, the bioactive glass is at least one selected from the group consisting of 45S5, S53P4, and 13-93.

In one embodiment, the bioactive glass is a mixture of 45S5 and 10-50 wt % of S53P4, based on a weight of 45S5.

A second aspect of the disclosure relates to a method for remineralizing teeth, the method comprising contacting the teeth with a toothpaste for 1-5 minutes, wherein the toothpaste comprises a bioactive glass and 10-50 wt % of a casein phosphopeptide, based on a weight of the bioactive glass, and a pH of the toothpaste when mixed with saliva is more than 7.3 and less than 8.

In one embodiment, the toothpaste is applied to a mouth tray and the teeth are contacted with the toothpaste present in the mouth tray.

In one embodiment, the bioactive glass comprises 40-80 wt % of silica and 20-50 wt % of calcium oxide, based on a weight of the bioactive glass.

In one embodiment, the bioactive glass is at least one selected from the group consisting of 45S5, S53P4, and 13-93.

In one embodiment, the bioactive glass is a mixture of 45S5 and 10-50 wt % of S53P4, based on a weight of 45S5.

In one embodiment, the toothpaste further comprises a fluoride source, and at least one of tri-calcium phosphate and amorphous calcium phosphate.

In one embodiment, the fluoride source is at least one selected from the group consisting of sodium fluoride, stannous fluoride, olaflur, and sodium monofluorophosphate.

In one embodiment, the toothpaste comprises 10-50 wt % of the tri-calcium phosphate, based on a weight of the fluoride source.

In one embodiment, the toothpaste comprises the tri-calcium phosphate and 10-50 wt % of the amorphous calcium phosphate, based on a weight of the tri-calcium phosphate.

### BRIEF DESCRIPTION OF THE DRAWINGS

FIG. 1 is a SEM micrograph of a dentin disc after 7 days of simulated brushing with Colgate® toothpaste (fluoride) (3,000× magnification, scale bar (the width of figure): 40 μm).

FIG. 2 is a SEM micrograph of a dentin disc that was brushed with Colgate® toothpaste (fluoride) after the citric acid challenge (6,000× magnification, scale bar (the width of figure): 20 μm).

FIG. 3 is a SEM micrograph of a dentin disc after 7 days of simulated brushing with Sensodyne® toothpaste (bioactive glass) (3,000× magnification, scale bar (the width of figure): 40 μm).

FIG. 4 is a SEM micrograph of a dentin disc that was brushed with Sensodyne® toothpaste (bioactive glass) after the citric acid challenge (6,000× magnification, scale bar (the width of figure): 20 μm).

FIG. 5 is a SEM micrograph of a dentin disc after 7 days of simulated brushing with Ultradex® toothpaste (hydroxyapatite) (3,000× magnification, scale bar (the width of figure): 40 μm).

FIG. 6 is a SEM micrograph of a dentin disc that was brushed with Ultradex® toothpaste (hydroxyapatite) after the citric acid challenge (6,000× magnification, scale bar (the width of figure): 20 μm).

FIG. 7 is a SEM micrograph of a dentin disc that was kept in a mixture of artificial saliva and Colgate® toothpaste (fluoride) for one week showing little to no tubule occlusion (6,000× magnification, scale bar (the width of figure): 20 μm).

FIG. 8 is a SEM micrograph of a dentin disc that was kept in a mixture of artificial saliva and Sensodyne® toothpaste (bioactive glass) for one week showing substantial tubule occlusion (6,000× magnification, scale bar (the width of figure): 20 μm).

FIG. 9 is a SEM micrograph of a dentin disc that was kept in a mixture of artificial saliva and Ultradex® toothpaste (hydroxyapatite) for one week showing considerable tubule occlusion (6,000× magnification, scale bar (the width of figure): 20 μm).

### DETAILED DESCRIPTION OF THE DISCLOSURE

Embodiments of the present disclosure will now be described more fully hereinafter with reference to the accompanying drawings, in which some, but not all embodiments of the disclosure are shown.

Within the description of this disclosure, where a numerical limit or range is stated, the endpoints are included unless stated otherwise. Also, all values and subranges within a numerical limit or range are specifically included as if explicitly written out. As used herein, the words “a” and “an” and the like carry the meaning of “one or more”.

The first aspect of the disclosure relates to a method for occluding dentin tubules, by contacting the dentin tubules with a toothpaste for 1-5 minutes to form occluded dentin tubules which are resistant to an acid such as a food acid and stomach acid. Exemplary food acids include, but are not limited to, citric acid, acetic acid, carbonic acid, lactic, malic acid, succinic acid, ascorbic acid, adipic acid, fumaric acid, tartaric acids, and mixtures thereof. The disclosed method occludes at least 80%, preferably at least 97%, more preferably at least 99% of the dentin tubules. As used herein, the term “resistant to an acid” means the occluded dentin tubules, after contacting with an acid, remain at least 10%, preferably at least 40%, preferably at least 80%, more preferably at least 90%, more preferably at least 97%, more preferably at least 99% occluded.

A percentage of the occluded dentin tubules can be calculated by dividing the number of tubules that are occluded by the total number of tubules that are present in a single micrograph and then multiplying it by 100. The number of occluded tubules and/or the extent of occlusion may be determined visually or by a software disclosed in patent application WO 2015050558 A1 (incorporated herein by reference in its entirety). Typically, the single micrograph is derived from a dentin disc which may be an extracted tooth or a portion thereof. Preferably, the micrograph is obtained by confocal microscopy, atomic force microscopy, or scanning electron microscopy. Images may be acquired by digitizing images from hard copies of micrographs obtained by confocal microscopy, atomic force microscopy, or scanning electron microscopy. Preferably, at least 3, preferably 10, more preferably 30 independent well-trained reviewers are contacted to assess the percentage of tubule occlusion from the micrographs. The reviewers may be trained with a standard set of micrographs of mixed samples containing fully open (no occlusion), partially (25%, 50%, or 75% occluded), and completely occluded dentin tubules. An agreement to the set standard may be quantified by Kappa analysis (Cohen J Psychol Bull 1960, 20:37-46, incorporated herein by reference in its entirety).

The dentin tubules may be contacted with the toothpaste at least once, preferably twice, more preferably thrice a day for 1-5 minutes, preferably 1-4 minutes, more preferably 1-3 minutes. This duration provides sufficient time for the ingredients in the toothpaste to interact with the dentin tubules and thereby allowing the ingredients to penetrate the dentin tubules before being washed away by saliva or rinsing. The contacting step may be carried out for at least 20 days, preferably at least 10 days, more preferably at least 7 days to occlude the dentin tubules. In one embodiment, the toothpaste is applied on a toothbrush and contacted with the dentin tubules. The toothbrush may have hard or soft bristles, and may be manual or battery-operated.

Preferably, the toothpaste may be applied to a mouth tray and the dentin tubules are contacted with the toothpaste present in the mouth tray. A mouth tray may be formed from thermoplastic polymers such as polyethylene and polypropylene and their derivatives and copolymers, silicone elastomers, polyurethanes and derivatives, polycaprolactams, polystyrene and derivatives, polybutadiene and derivatives, polyisoprene and derivatives, and polymethacrylate and derivatives. Preferably, the mouth tray is formed from polyethylene. The mouth tray may fit either the entire upper or lower rows of the teeth, or both. The mouth tray may be made by first taking a cast of the teeth and gum area of a user and then allowing the cast to set. Secondly, the thermoplastic polymer film is placed over the cast and vacuum is applied to force the thermoplastic polymer film to form the mouth tray in the shape of the teeth and gum margin of the user. The toothpaste may be applied to the tray, preferably 0.5-3 ml of toothpaste per tray, preferably 0.5-2 ml, more preferably 0.75-1.25 ml. In one embodiment, the user may apply a teeth whitening paste to the mouth tray so that the teeth are whitened and treated for sensitivity simultaneously. The teeth whitening paste may comprise active ingredients such as hydrogen peroxide, carbamide peroxide. The user may wear the mouth tray with the toothpaste on the lower row of teeth, the upper row of teeth, or both simultaneously for the aforementioned duration. After which, the user may remove the mouth tray(s) and rinse the mouth to get rid of any toothpaste residue.

In another embodiment, the toothpaste is applied to a strip and the dentin tubules are contacted with the toothpaste on the strip. The toothpaste and optionally the aforementioned teeth whitening paste may be pre-applied to the strip or applied to the strip by the user. The amount of toothpaste ranges from 0.05-0.8 ml, preferably 0.1-0.5 ml, more preferably 0.3-0.5 ml. The strip may comprise materials such as polymers, natural and synthetic wovens, non-wovens, foil, paper, rubber, and combinations thereof. The strip may be a single layer of material or a laminate of more than one layer. Exemplary polymers include, but are not limited to, polyethylene, ethylvinylacetate, ethylvinyl alcohol, polyesters such as Mylar® manufactured by DuPont, fluoroplastics such as Teflon® manufactured by DuPont, and combinations thereof. Preferably, the strip is formed from polyethylene. The strip is preferably of a size that fits either the entire upper or lower rows of teeth. The length of the strip may range from 2-12 cm, preferably 4-12 cm, more preferably 6-12 cm. The strip may cover only a surface of the teeth. In a preferred embodiment, the strip covers both surfaces of the tooth. The width of the strip may range from 0.5-4 cm, preferably 1-4 cm, more preferably 1-3 cm. The thickness of the strip may range from 20-1,500 μm, preferably 50-1,000 μm, more preferably 800-900 μm.

The contacting step may involve at least one of the aforementioned steps. For example, the user may contact the dentin tubules with toothpaste by brushing followed by wearing the mouth trays. In another embodiment, the user applies the strip and then puts on the mouth tray.

In another embodiment, the toothpaste is applied in the form of a gel or hydrogel. The gel form permits extended contact of water-based compositions with the teeth. Preferably, the hydrogels contain one or more polymers such as polyacrylic acid, sodium polyacrylate, polyacrylamide copolymer, ethylene maleic anhydride copolymer, cross-linked carboxymethylcellulose, polyvinyl alcohol copolymer, cross-linked polyethylene oxide, and starch grafted copolymer of polyacrylonitrile. In another embodiment, the composition is mixed with hyaluronic acid gel and used as a short term pretreatment or extended treatment for the teeth.

The toothpaste may be in the form of a paste or a gel. In one embodiment, a toothpaste comprising fluoride is in the form of a gel and can be left on the teeth to keep the fluoride longer in the mouth thereby promoting remineralization of teeth. The toothpaste may be contacted with the teeth with the toothbrush, the mouth tray and/or the strip. In an alternative embodiment, the toothpaste is in the form of an aqueous solution comprising water and optionally an alcohol such as ethanol and isopropanol. The alcohol may be present in an amount ranging from 5-40 vol %, preferably 10-30 vol %, more preferably 10-20 vol %, based on a total volume of the toothpaste. In another embodiment, the toothpaste is in the form of a solution comprising the alcohol and does not contain water, e.g., or alternately a non-aqueous solution, suspension or dispersion.

The presence of dental plaque may interfere with the occlusion of dentin tubules and reduce the effectiveness of the toothpaste. In one embodiment, the user chews on a disclosing tablet after contacting the teeth with the toothpaste. Alternatively, the user may rinse the mouth with a disclosing solution, apply a disclosing swab to the surfaces of the teeth, or use disclosing floss. The disclosing tablet/solution/swab/floss stains dental plaque, indicating the problem areas which need more toothpaste. The user may then repeat the contacting step to remove the plaque and facilitate the occlusion of dentin tubules. Exemplary disclosing tablet/solution/swab/floss includes products sold under the brands of Trace, PlaqSearch, and Plak Smacker.

The toothpaste comprises theobromine and at least one of a bioactive glass and hydroxyapatite. A ratio of the theobromine concentration to the concentration of the at least one of bioactive glass and hydroxyapatite may range from 5:95 to 95:5, preferably 5:95 to 80:20, preferably 5:95 to 70:30, preferably 5:95 60:40, preferably 5:95 to 50:50, preferably 5:95 to 40:60, preferably 5:95 to 30:70, preferably 5:95 to 20:80, more preferably 5:95 to 10:90. In other embodiments, the toothpaste comprises 10-30 wt % theobromine, preferably 10-20 wt %, more preferably 14-16 wt %, based on a total weight of the at least one of bioactive glass and hydroxyapatite. In some embodiments, the toothpaste comprises the bioactive glass and 5-95 wt % of hydroxyapatite, preferably 10-50 wt %, more preferably 20-30 wt %, based on a weight of the bioactive glass. In at least one embodiment, the toothpaste comprises hydroxyapatite, and further comprises 10-50 wt % of fluorapatite, preferably 20-40 wt %, more preferably 30-40 wt %, based on a weight of hydroxyapatite.

Theobromine may be present in the toothpaste as a neutral compound, an amine salt or a double salt thereof (e.g., with alkali metal salts or alkaline earth metal salts of organic acids, for example alkali or alkaline earth metal salts of acetic, gluconic, benzoic, or salicylic acid). The double salts may be prepared either to make the theobromine more water soluble, or to make insoluble complexes. Suitable amine salts of theobromine include the acid addition salts including: (1) pharmaceutically acceptable inorganic salts, such as sulfate, nitrate, phosphate, borate, hydrochloride and hydrobromide, and (2) pharmaceutically acceptable organic acid addition salts such as acetate, tartrate, maleate, citrate, succinate, benzoate, ascorbate, methanesulfonate, a-keto glutarate, a-glycerophosphate and glucose-i-phosphate. Preferably, the acid addition salt is a hydrochloride salt.

Theobromine is preferably in a pharmaceutically acceptable form. By pharmaceutically acceptable form is meant, inter alia, of a pharmaceutically acceptable level of purity excluding normal pharmaceutical additives, such as diluents and carriers, and including no material considered toxic at normal dosage levels. A pharmaceutically acceptable level of purity will generally be at least 50%, preferably 75%, more preferably 90%, and still more preferably 95%, based on the weight of the theobromine.

The toothpaste may also comprise a structural analog of theobromine, which is represented by formula (I) and contains at least one functional group that enhances the ability of the structural analog to occlude dentin tubules:

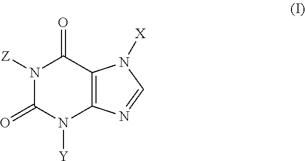

where X, Y, and Z are each independently an optionally substituted alkyl, an optionally substituted arylalkyl, The optionally substituted arylalkyl may be, but is not limited to, benzyl, phenethyl, and phenylpropyl. As used herein, the term “substituted” refers to at least one hydrogen atom that is replaced with a non-hydrogen group, provided that normal valencies are maintained and that the substitution results in a stable compound. When a compound or a X, Y, and Z group are noted as “optionally substituted”, the substituents are selected from the exemplary group including, but not limited to, halo, hydroxy, alkoxy, oxo, alkanoyl, aryloxy, alkanoyloxy, amino, alkylamino, arylamino, arylalkylamino, disubstituted amines (e.g. in which the two amino substituents are selected from the exemplary group including, but not limited to, alkyl, aryl, or arylakyl), alkanylamino, aroylamino, aralkanoylamino, substituted alkanoylamino, substituted arylamino, substituted aralkanoylamino, thiol, alkylthio, arylthio, arylalkylthio, alkylthiono, arylthiono, aryalkylthiono, alkylsulfonyl, arylsulfonyl, arylalkylsulfonyl, sulfonamido (e.g. —SO₂NH₂), substituted sulfonamide, nitro, cyano, carboxy, carbamyl (e.g. —CONH₂), substituted carbamyl (e.g. —CONHalkyl, —CONHaryl, —CONHarylalkyl or cases where there are two substituents on one nitrogen from alkyl, aryl, or arylalkyl), alkoxycarbonyl, aryl, substituted aryl, guanidine, heterocyclyl (e.g. indolyl, imidazoyl, furyl, thienyl, thiazolyl, pyrrolidyl, pyridyl, pyrimidiyl, pyrrolidinyl, piperidinyl, morpholinyl, piperazinyl, homopiperazinyl and the like), substituted heterocyclyl and mixtures thereof and the like. The substituents may be either unprotected, or protected as necessary, as known to those skilled in the art, for example, as taught in Greene, et al., “Protective Groups in Organic Synthesis”, John Wiley and Sons, Second Edition, 1991, hereby incorporated by reference in its entirety).

The toothpaste may also comprise 1-99 wt % of the structural analog of theobromine, preferably 1-50 wt %, more preferably 1-10 wt %, based on a weight of theobromine. Exemplary structural analogs of theobromine include, but are not limited to, theophylline and paraxanthine. The toothpaste may comprise theophylline, paraxanthine, or both. In one embodiment, the toothpaste comprises paraxanthine and 1-99 wt % of theophylline, preferably 1-30 wt %, more preferably 1-10 wt %, based on a weight of paraxanthine. Theobromine, theophylline, and paraxanthine are natural metabolites of caffeine and form (in weight ratio of about 3:1:21) when caffeine is broken down by cytochrome P450. In the production of the toothpaste, caffeine may be employed as one of the raw materials, and may be broken down by cytochrome P450 to yield theobromine, theophylline, and paraxanthine, which are then added to the toothpaste. In one embodiment, the toothpaste comprises paraxanthine, 15-20 wt % of theobromine, 4-6 wt % of theophylline, based on the weight of paraxanthine.

In some embodiments, the toothpaste comprises an apatite derivative such as fluorapatite, carbonated fluorapatite, hydroxyapatite, carbonated hydroxyapatite, or mixtures thereof. In some embodiments, the apatite derivative is silanized by contacting an apatite derivative particle with a silane coupling agent such as glycidoxypropyl trimethoxy silane, γ-methacryloxypropyltrimethoxy silane, N-(β-aminoethyl)-γ-aminopropyltrimethoxy silane, and mixtures thereof (Deng C et al. J Mater Sci Mater Med 2010 21:3059-3064, incorporated herein by reference in its entirety). An amount of silicon may range from 1-49 wt %, preferably 5-30 wt %, more preferably 5-10 wt %, based on a total weight of the silanized apatite derivative. The apatite derivative may be amorphous. Preferably, the apatite derivative is crystalline, and may have a nanorod shape. The apatite derivative particle may have a length ranging from 500 nm to 20 μm, preferably 500 nm to 5 μm, more preferably 500 nm to 2 μm. A cross section of the apatite derivative particle may range from 10 nm to 3 μm, preferably 10-500 nm, more preferably 20-100 nm.

In some embodiments, the toothpaste comprises bioactive glass. As used herein, the term “bioactive glass” refers to an inorganic glass material comprising an oxide of silicon as its major component and is capable of bonding with growing tissue when reacted with physiological fluids. For example, a bioactive glass is a glass composition that will form a layer of hydroxycarbonate apatite in vitro when placed in a simulated body fluid. A bioactive glass is also bioacceptable such that it does not trigger an adverse immune response in the body, such as in the oral cavity.

The bioactive glass may comprise 40-55 wt % of silica, preferably 40-50 wt %, more preferably 43-46 wt %, based on a total weight of the bioactive glass. The bioactive glass may further comprise 20-50 wt % calcium oxide, preferably 20-30 wt %, more preferably 25-30 wt %. In some embodiments, the bioactive glass comprises 10-35 wt % sodium oxide, preferably 10-25 wt %, more preferably 10-15 wt %. In other embodiments, the bioactive glass comprises 2-8 wt % of phosphorus pentoxide, preferably 3-8 wt %, more preferably 6-8 wt %.

In alternative embodiments, the bioactive glass comprises up to 25 wt % of calcium fluoride, preferably up to 15 wt %, more preferably up to 8 wt %. In one embodiment, the bioactive glass comprises up to 10 wt % of boron trioxide, preferably up to 8 wt %, more preferably up to 3 wt %. In one embodiment, the bioactive glass comprises up to 8 wt % of potassium oxide, preferably up to 3 wt %, more preferably up to 1 wt %. In another embodiment, the bioactive glass comprises up to 5 wt % of magnesium oxide, preferably up to 3 wt %, more preferably up to 0.5 wt %.

In one embodiment, the bioactive glass is calcium sodium phosphosilicate. In other embodiments, the bioactive glass may be 45S5, S53P4, 13-93, or mixtures thereof. In one embodiment, the toothpaste comprises 45S5 and 10-50 wt % of S53P4, preferably 30-50 wt %, more preferably 45-50 wt %, based on a weight of 45S5.

The bioactive glass is in the form of a particle with a median particle size ranging from 0.5-710 μm, preferably 0.5-90 μm, more preferably 0.5-20 μm. In one embodiment, the toothpaste comprises bioactive glass particles of one median particle size. In another embodiment, the toothpaste comprises bioactive glass particles of at least two median particle sizes. The smaller bioactive glass particle may have a median particle size ranging from 0.5-20 μm, preferably 0.5-10 μm, more preferably 0.5-2 μm so that they will fit inside dentin tubules. A diameter of the dentin tubules may range from 0.1-10 μm, preferably 0.1-5 μm, more preferably 0.5-3 μm. The larger bioactive glass particle may have a median particle size ranging from 1-710 μm, preferably 10-90 μm, more preferably 30-90 μm. The larger bioactive glass particles may adhere to tooth structure and act as ionic reservoirs, providing additional calcium and phosphate ions so that the deposition of the calcium phosphate layer begun by the small particles can continue. The toothpaste may comprise 10-90 wt % of the larger bioactive glass particle, preferably 10-40 wt %, more preferably 10-20 wt %, based on a weight of the smaller bioactive glass particle.

The second aspect of the disclosure relates to a method for remineralizing teeth by contacting teeth with a toothpaste which comprises the aforementioned bioactive glass and 10-50 wt % of a casein phosphopeptide, preferably 10-30 wt %, more preferably 10-20 wt %, based on a weight of the bioactive glass. Remineralization of the teeth may be assessed by the aforementioned microscopy techniques, preferably scanning electron microscopy. As used herein, the term “remineralization” is the formation of hydroxycarbonate apatite on a tooth surface. The toothpaste has a pH in a range of more than 7.3 to less than 8, 7.5-7.9, preferably 7.7-7.9 when mixed with saliva (either artificial or natural). The method for remineralizing teeth may involve the aforementioned steps of the method for occluding dentin tubules.

In some embodiments, the toothpaste further comprises a fluoride source, and at least one of tri-calcium phosphate and amorphous calcium phosphate. Exemplary sources of fluoride include, but are not limited to, sodium monofluorophosphate, stannous fluoride, alkali fluorides, such as sodium fluoride, potassium fluoride, lithium fluoride, and ammonium fluoride, tin fluoride, indium fluoride, zirconium fluoride, copper fluoride, nickel fluoride, palladium fluoride, fluorozirconates, such as sodium fluorozirconate, potassium fluorozirconate, ammonium fluorozirconate, and tin fluorozirconate, fluorosilicates, fluoroborates, fluorostannites, and mixtures thereof. Organic fluorides, such as olaflur, may also be present in the toothpaste. An amount of fluoride in the toothpaste may be up to 10,000 ppm, preferably up to 5,000 ppm, more preferably up to 2,000 ppm. In one embodiment, the toothpaste comprises 10-50 wt % of the tri-calcium phosphate, preferably 10-30 wt %, more preferably 10-20 wt %, based on a weight of the fluoride. In another embodiment, the toothpaste comprises tri-calcium phosphate and 10-50 wt % of the amorphous calcium phosphate, preferably 30-50 wt %, more preferably 40-50 wt %, based on a weight of the tri-calcium phosphate.

Tricalcium phosphate may be α-tricalcium phosphate or β-tricalcium phosphate, preferably β-tricalcium phosphate, which has a higher solubility in living systems than α-tricalcium phosphate. Tricalcium phosphate may be in the form of a particle with a median particle size ranging from 100 nm to 20 μm, preferably 300 nm to 5 μm, more preferably 300-800 nm.

Amorphous calcium phosphate lacks the long-range, periodic atomic scale order of crystalline calcium phosphates, and hence it readily dissolves into the saliva to form stable hydroxyapatite and/or fluorapatite. The amorphous calcium phosphate may be in the form of a particle with a median particle size ranging from 10-100 nm, preferably 30-100 nm, more preferably 70-100 nm. Amorphous calcium phosphate may have a molar calcium/phosphate ratio ranging from 1.1:1 to 1.8:1, preferably 1.4:1 to 1.7:1, more preferably 1.4:1 to 1.6:1.

Biomolecules, such as casein phosphopeptide and γ-polyglutamic acid, may form a complex with amorphous calcium phosphate thereby improving its efficacy in preventing tooth decay. An amount of the biomolecule may range from 1-99 wt %, preferably 20-80 wt %, more preferably 40-60 wt %, based on a weight of amorphous calcium phosphate.

The phosphopeptide, preferably casein phosphopeptide, may be made synthetically by chemical synthesis or genetic engineering or extracted from naturally occurring materials. For example, the phosphopeptide may be obtained by hydrolyzing or digesting (either chemical or proteolytic) a protein or by tryptic digestion of casein or other phospho-acid rich proteins such as phosphitin, or by chemical or recombinant synthesis, provided that the phosphopeptide comprises the core sequence -Ser(P)-Ser(P)-Ser(P)-Glu-Glu-. In a preferred embodiment, casein is digested with trypsin, pepsin, chymotrypsin, papain, thermolysin or pronase, preferably trypsin. In addition, extracting the phosphopeptide from casein and in particular from α-casein or β-casein is more economical. Further, phosphoproteins in cereals, nuts and vegetables particularly in bran husks or sheaths (rice, wheat, oat, barley or rye brans) may be used to produce the phosphopeptide. Soybean and meat contain phosphoproteins which may be of use in obtaining the phosphopeptide above.

In one embodiment, the biomolecule is γ-polyglutamic acid and may be the acid form, the salt form or a mixture thereof. Exemplary γ-polyglutamate salts include, but are not limited to, sodium γ-polyglutamate, potassium γ-polyglutamate, calcium γ-polyglutamate, magnesium γ-polyglutamate, and zinc γ-polyglutamate.

The present embodiments are being described with reference to specific example embodiments and are included to illustrate but not limit the scope of the invention.

### Example 1 Materials and Methods

The study to evaluate dentin tubule occlusion and remineralization competence of toothpastes was designed in accordance with the ethical standards specified in the 1964 Helsinki Declaration and its later amendments and was approved by the Institutional Review Board of University of Dammam (Ref.: IRB-2014-02-032). All ethical protocols were strictly followed.

Three toothpastes containing fluoride (Colgate®, Colgate-Palmolive Co., Saudi Arabia), bioactive glass (Sensodyne® Repair and Protect, Glaxo Smith Kline, Weybridge, Surrey, UK) and hydroxyapatite (Ultradex Recalcifying and Whitening Toothpaste®, Periproducts Limited, Ruislip Middlesex, UK) were selected for this study.

### Example 2 Dentin Disc Preparation

Sixty non-carious extracted third molars belonging to healthy patients were collected from the Oral and Maxillofacial Surgery Clinic of the College of Dentistry, University of Dammam. They were preserved in 10% formalin at room temperature for no longer than a month before use. Sixty dentin discs of 1.5 mm (±0.2 mm) were prepared by cutting teeth mesio-distally over the cemento-enamel junction using a water-cooled diamond saw (Isomet® 5000 Linear Precision Saw, Buehler Ltd, IL, USA) at a blade speed of 3,500 rpm and a feed rate of 15 mm/min. Occlusal enamel was also removed in a similar manner to expose the mid dentin. The upper surfaces of the dentin discs were marked and the other unmarked surfaces were wet ground with 600-grit silicon carbide paper for 30 s under water irrigation. The unmarked dentin disc surfaces were etched with ethylene-diamine-tetraacetic acid (EDTA) for 1 min and then washed with distilled water for 1 min to open the dentin tubules and remove any organic material.

### Example 3 Preparing and Experiments with Artificial Saliva (AS)

After washing with distilled water, dentin discs were stored at room temperature in artificial saliva that was freshly prepared according to the composition proposed by Fusayama et al. (Fusayama T, Katayori T, Nomoto S. J Dent Res 1963, 42:1183-97, incorporated herein by reference in its entirety) (Table 1). The pH of the newly prepared artificial saliva was 5.2, which was checked using a pH meter (Precisa, model pH 900, Switzerland), and was adjusted by adding 1 M of sodium hydroxide (NaOH) until a pH of 7.2 was obtained.

TABLE 1
Composition of artificial saliva

| Chemical | Quantity | |
|---|---|---|
| NaCl | 0.400 | g |
| KCl | 0.400 | g |
| NaH₂PO₄•H₂O | 0.69 | g |
| CaCl₂•H₂O | 0.795 | g |
| Na₂S•9H₂O | 0.005 | g |
| Deionized water | 1000 | mL |

After immersion of 2 g of toothpaste in artificial saliva, it was observed that the pH of the artificial saliva containing Colgate® toothpaste (fluoride) became 7.8, that of the artificial saliva containing the Sensodyne® toothpaste (bioactive glass) increased to 7.6, and that of the artificial saliva containing Ultradex® toothpaste (hydroxyapatite) increased to 7.3 (Table 2).

TABLE 2
pH of AS before and after the addition of various toothpastes.

| pH of AS before | | pH of AS after |
|---|---|---|
| immersion of | Toothpaste brand | immersion of |
| toothpastes | immersed in AS | toothpastes |
| 7.2 | Colgate ® | 7.8 |
| 7.2 | Sensodyne ® | 7.6 |
| 7.2 | Ultradex ® | 7.3 |

### Example 4 Tooth Brushing Experiments

Forty dentin discs (out of 60) were randomly selected for brushing experiments and were divided into five groups, each containing eight specimens that received the following treatments: group 1 (control)—EDTA-etched discs, group 2—EDTA-etched discs brushed with distilled water, group 3—EDTA-etched discs brushed with 1.0 g Colgate® toothpaste, group 4—EDTA-etched discs brushed with 1.0 g Sensodyne® toothpaste, and group 5-EDTA-etched discs brushed with 1.0 g Ultradex® toothpaste. The brushing experiments were conducted for 2 min twice a day (at 8 am and 4 pm) for 7 days utilizing soft bristled toothbrushes (Oral B®, Pro-Flex™, USA) inside a toothbrush simulation machine (Toothbrush simulator, model ZM-3.8, Germany), under a continuous loading of 150 g for 150 strokes/min. After every brushing cycle, the discs were washed with distilled water for 1 min and returned to their respective labelled container containing artificial saliva at 37° C., which was replaced every 24 h. The toothbrushes were also washed with distilled water for 1 min after every brushing cycle.

### Example 5 Citric Acid Challenge

Four out of eight dentin discs from each group were randomly selected for the citric acid challenge. Dentin discs were immersed into 6 wt % citric acid (pH 2.2) for 1 min to evaluate the resistance of particles occluding dentin tubules against the acid challenge. The discs were then washed with distilled water for 2 min and dried in a desiccator (Dry-Keeper™ Desiccator Cabinets, Cole-Parmer®, UK) for 24 h before SEM analysis.

### Example 6 Characterizing Remineralization Potential

The remaining twenty dentin discs were randomly divided into four groups, with each group containing five discs. The discs were kept in an individual labelled container containing 1 g of toothpaste per 5 mL of artificial saliva. A total of 10 mL of this mixture was utilized. The discs received the following treatment: group A (control)—EDTA-etched discs kept in distilled water, group B—EDTA-etched discs kept in a mixture of artificial saliva (pH 7.2) and 2 g Colgate® toothpaste (fluoride), group C—EDTA-etched discs kept in a mixture of artificial saliva and 2 g Sensodyne® toothpaste (bioactive glass), and group D—EDTA-etched discs kept in a mixture of artificial saliva and 2 g Ultradex® toothpaste (hydroxyapatite). The dentin discs were left in the mixture for one week at 37° C. After a week, the discs were washed with distilled water for 1 min and dried in a desiccator (Dry-Keeper™ Desiccator Cabinets, Cole-Palmer, UK) for 24 h before SEM analysis. The pH of the leftover mixture/solution was analyzed using a pH meter.

With regards to remineralization, all mixtures showed a pH elevation. However, the highest pH increase was demonstrated by the mixture of artificial saliva+Colgate® toothpaste, followed by artificial saliva+Sensodyne® toothpaste and artificial saliva+Ultradex® toothpaste. The processes of demineralization (when the pH of the oral cavity becomes acidic) and remineralization (when the pH of the oral cavity becomes alkaline) occur continuously in the oral cavity, and usually, there is an equilibrium present between these two (Featherstone J D B. Aust Dent J 2008, 53:286-91, incorporated herein by reference in its entirety). An increase in the pH with the mixture of artificial saliva+toothpaste shows the buffering capacity of a toothpaste. This is not only useful in neutralizing the acids that are released by bacteria in dental plaque, but is also useful in normalizing the pH of the saliva after an acid attack.

When the dentin discs (immersed in the respective mixture of toothpaste and artificial saliva for 7 days) were observed under SEM, the highest tubule occlusion was revealed by the mixture of artificial saliva+Sensodyne® toothpaste followed by artificial saliva+Ultradex® toothpaste. Very little occlusion was demonstrated by the mixture of artificial saliva+Colgate® toothpaste. Immersion of dentin discs into the artificial saliva+toothpaste mixture was performed to simulate realistic in vivo conditions that are present for dentin remineralization and tubule occlusion. The presence of abundant toothpaste particles on the dentin surface could be attributed to the fact that both Sensodyne® and Ultradex® toothpastes increase the quantity of calcium and phosphate ions in the saliva, which results in the formation of a calcium-phosphate (Ca—P) layer on the dentin surfaces, leading to physical occlusion of dentin tubules (Reynolds E C. Aust Dent J 2008, 53:268-73, incorporated herein by reference in its entirety).

### Example 7 Statistical Analysis

Descriptive statistics were estimated using the Statistical Package for the Social Sciences (SPSS) software (version 19.0, SPSS Inc., Chicago, Ill., USA). The Wilcoxon signed-rank test was used to compare the mean percentages of tubule occlusion pre- and post-citric acid challenge that was demonstrated by individual toothpastes with various active ingredients. The level of significance was set at p<0.05.

Table 3 presents the mean percentages of tubule occlusion that were achieved by various toothpastes after 7 days of brushing and after the citric acid challenge. The Wilcoxon signed-rank test revealed a significant difference (p<0.05) in tubule occlusion before and after the acid treatment for groups 3 and 4. In contrast, in group 5, although some tubules became unoccluded after acid treatment, the difference was not significant.

TABLE 3
Percentage of tubule occlusion pre- and post-citric
acid challenge demonstrated by various toothpastes.

| | After 7 days | |
|---|---|---|
| | of brushing | |
| | (pre-citric acid | Post-citric |
| Group | challenge) | acid challenge |
| Group 3 (Fluoride) | 97.8 ± 0.84 | 0.4 ± 0.89 [symbol] |
| Group 4 (Bioactive glass) | 99.8 ± 0.45 | 97.6 ± 1.14 [symbol] |
| Group 5 (Hydroxyapatite) | 100 ± 0.00 | 99.8 ± 0.45 |

Values expressed as (%) were reported as the mean ± standard deviation. [symbol]
Wilcoxon signed-rank test, significant at p < 0.05. [symbol]
indicates data missing or illegible when filed

### Example 8 SEM Analysis

Dentin discs were mounted on stubs, coated with a thin layer of gold, and analyzed in an SEM (FEI, Inspect F50, The Netherlands) for occlusion of dentin tubules using an electron mode of 15 kV. Micrographs were taken from the central portion of the dentin discs at 3,000×, 6,000×, and 10,000× magnifications. The percentage of the occluded dentin tubules was calculated by dividing the number of tubules that were occluded by the total number of tubules that were present in a single micrograph and then multiplying it by 100. Three independent well-trained blind reviewers were contacted to assess the percentage of tubule occlusion from the SEM images. The reviewers were requested to report the percentage of tubule occlusion by assessing every tubule in the individual SEM image and reporting the mean percentage of occluded tubules in individual SEM micrographs based on the following criteria: No occlusion=0%, 1/4=25% occluded, 2/4=50% occluded, 3/4=75% occluded, and 4/4=100% occluded.

An SEM micrograph of dentin discs from the control and distilled water groups showed no tubule occlusion after one week of brushing experiments, and all dentin tubules appeared unfilled (not shown).

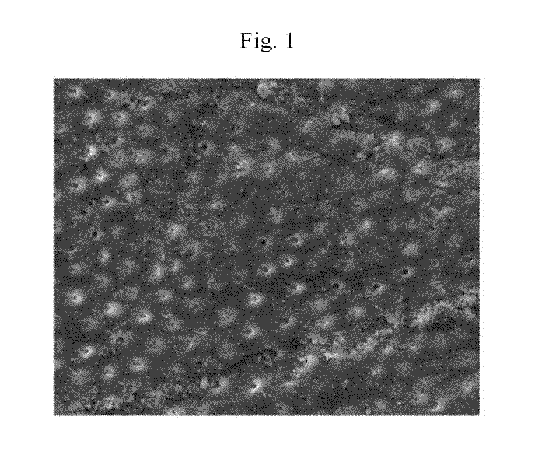

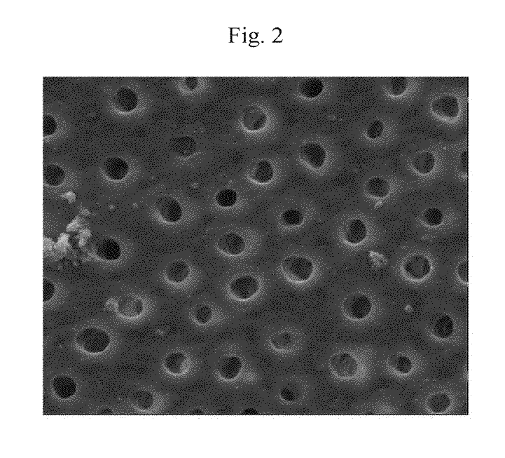

Images from group 3, in which the discs were brushed with Colgate® toothpaste (fluoride), showed tubule occlusion after the brushing experiments and before the citric acid challenge (FIG. 1). However, after the citric acid challenge, most of the tubules appeared to be unoccupied by toothpaste particles (FIG. 2).

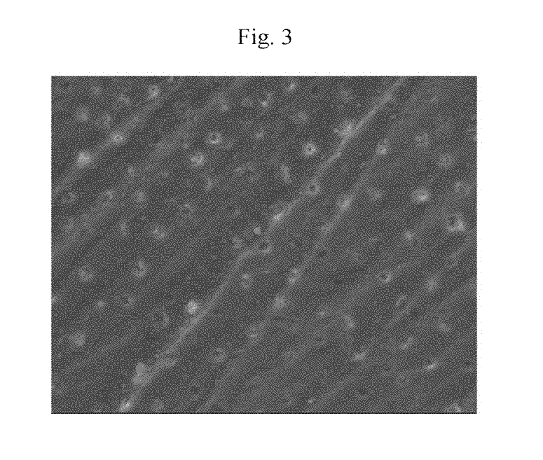

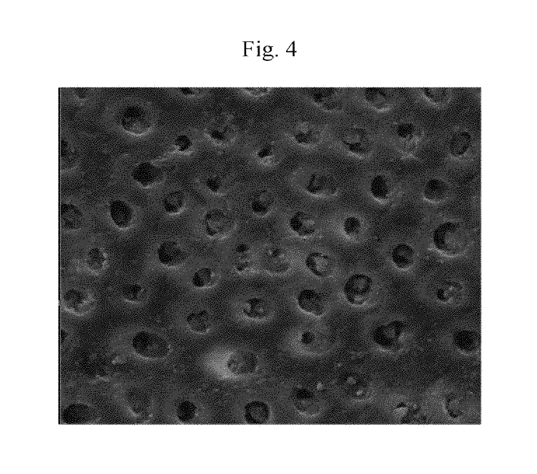

Compared with group 3, micrographs from group 4 (FIG. 3) showed superior tubule occlusion that was achieved when the discs were brushed with Sensodyne® toothpaste (bioactive glass) (before the acid challenge). The majority of the tubules remained blocked even after acid exposure (FIG. 4).

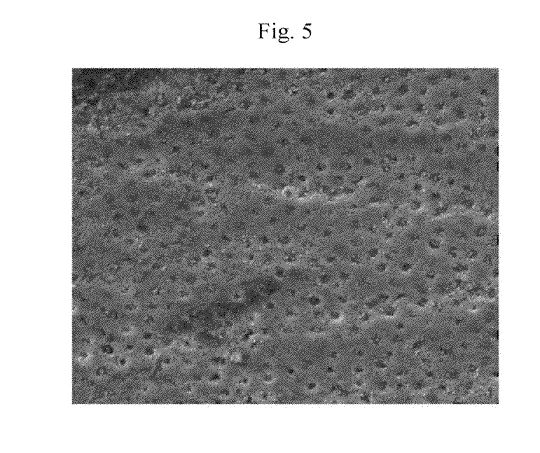

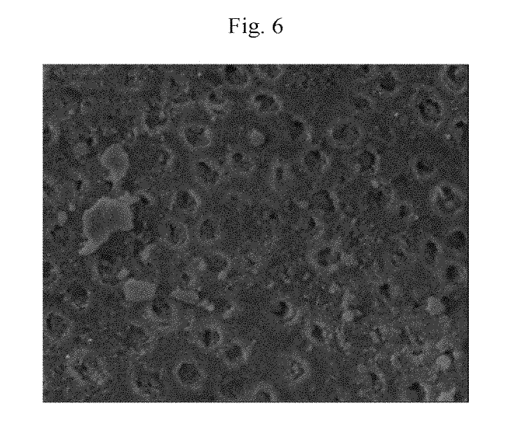

High-magnification SEM micrographs of group 5, in comparison with the micrographs of groups 3 and 4, indicated maximum tubule occlusion when the dentin discs were brushed with Ultradex® toothpaste (hydroxyapatite), both pre- (FIG. 5) and post-citric acid challenge (FIG. 6). Clear visual differences were present in the post-citric acid challenge SEM images of dentin discs for group 4 and 5. After the citric acid challenge, the precipitation layer on top of the dentin discs belonging to group 4 was washed away, but for group 5, the precipitation layer formed by Ultradex® toothpaste (hydroxyapatite) particles was still present.

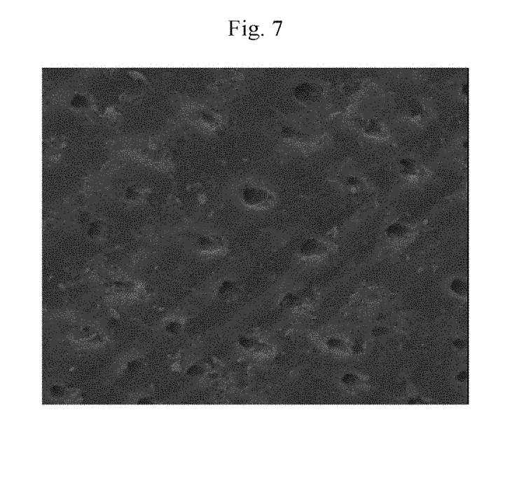

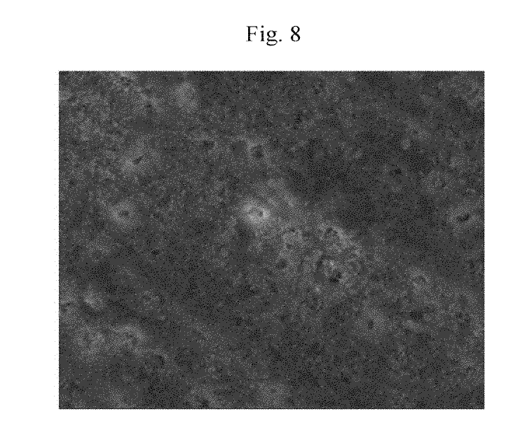

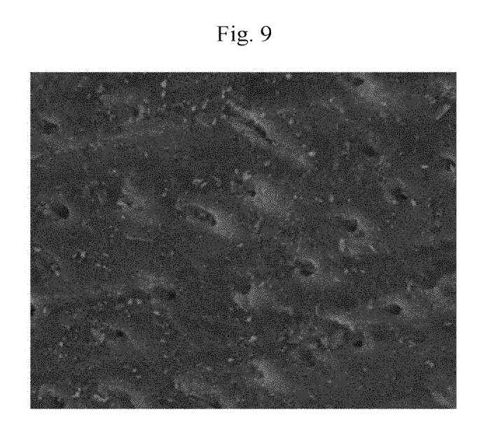

SEM photomicrographs taken after immersion of dentin discs in distilled water (group A) presented no tubule occlusion (not shown). The micrographs of the discs kept in a mixture of artificial saliva and Colgate® toothpaste (fluoride) (group B) showed little to no tubule occlusion (FIG. 7) although toothpaste particles were visible on the disc surfaces. However, SEM micrographs of dentin discs kept in the mixture of artificial saliva and Sensodyne® toothpaste (bioactive glass) (FIG. 8) and the mixture of artificial saliva and Ultradex® toothpaste (hydroxyapatite) (FIG. 9) exhibited greater surface coverage, presence of amorphous particles, and considerable tubule occlusion on treated disc surfaces.

Fluoride is a remineralizing agent that has the ability to form fluorapatite in the crystal lattice of enamel, which is more resistant to acid challenges (Lata S, Varghese N O, Varughese M J. J Conserv Dent 2010, 13(1):42-6, incorporated herein by reference in its entirety). In addition, fluoride has the ability to block dentin tubules by precipitation (Furseth R. Acta Odontol Scand 1970, 28(6):833-50, incorporated herein by reference in its entirety). Bioactive glass is a biocompatible material that is traditionally used for osteogenesis, but recently, its use in the field of dentistry has been encouraged because of the resemblances between bone and dentin (Hench L L, Splinter R J, Allen W C, Greenlee T K. J Biomed Mater Res 1971, 5(6):117-41; and Farooq I, Imran Z, Farooq U, Leghari A, Ali H. World J Dent 2012, 3(2):199-201, each incorporated herein by reference in their entirety). Hydroxyapatite is the core constituent of hard tissues in the human body (Zakaria S M, Sharif Zein S H, Othman M R, Yang F, Jansen J A. Tissue Eng Part B Rev 2013, 19(5):431-41, incorporated herein by reference in its entirety). Its superior remineralization potential has encouraged its use in dentistry, particularly as an active ingredient in dentifrices (Al-Sanabani S J, Madfa A A, Al-Sanabani A F. Int J Biomater 2013, 2013:1-12, incorporated herein by reference in its entirety).

After one week of brushing experiments, the results of the present study indicate that the highest tubule occlusion competence was achieved by using Ultradex® toothpaste (hydroxyapatite) (post-citric acid challenge). This was followed by Sensodyne® toothpaste (bioactive glass), whereas Colgate® toothpaste (fluoride) presented with little to no tubule occlusion under SEM. After exposure of the dentin discs to 6% citric acid in the present study, the SEM micrographs from all groups showed empty dentin tubules except for the dentin discs that were treated with Sensodyne® (bioactive glass) and Ultradex® toothpaste (hydroxyapatite). This could be explained by the remineralization and occlusion potential of these dentifrices.

All dentifrices showed considerable tubule occlusion after one week of simulated brushing before the acid challenge. However, after exposure to citric acid, particles of Colgate® toothpaste (fluoride) were almost completely washed away from the tubules whereas only a slight reduction in Ultradex® toothpaste (hydroxyapatite) particles was noticed. It has been demonstrated that hydroxyapatite particles (consisting of calcium and phosphate) have the ability to adsorb onto the tooth surface and inhibit dissolution (Hornby K, Evans M, Long M, Joiner A B, Laucello M, Salvaderi A. Int Dent J 2009, 59:325-31, incorporated herein by reference in its entirety). This most likely led to greater tubule occlusion achieved by Ultradex® toothpaste (hydroxyapatite) compared with other toothpastes in this study.

After Ultradex® toothpaste (hydroxyapatite), Sensodyne® toothpaste (bioactive glass) demonstrated better tubule occlusion than the other groups. Although SEM images reveal that the precipitation layer formed by Sensodyne® toothpaste (bioactive glass) was washed away after encountering citric acid, many of the tubules remained occluded (few completely and few partially) with toothpaste particles. This shows good retentive properties of Sensodyne® toothpaste (bioactive glass) particles. Analyzing the extent of the penetration of particles inside dentin tubules was not part of the present study, but it could form a solid basis for future work in this area. bioactive glass (consisting of calcium sodium phosphosilicate) also has the ability to physically occlude dentin tubules because its presence in an aqueous solution like saliva encourages the release of sodium ions and raises the pH, which leads to increased precipitation and development of a calcium hydroxyapatite layer on the dentin surface (Milleman L J, Milleman R K, Clark E C, Mongiello A K, Simonton C T, Proskin M H. Am J Dent 2012, 25(5):262-8, incorporated herein by reference in its entirety).

1: A method for occluding dentin tubules, the method comprising:
contacting the dentin tubules with a toothpaste for 1 to 5 minutes thereby forming occluded dentin tubules which are resistant to an acid,
wherein the toothpaste comprises theobromine, a bioactive glass, and hydroxyapatite, a weight ratio of theobromine to the bioactive glass and hydroxyapatite ranges from 5:95 to 95:5, the acid is citric acid or acetic acid, the hydroxyapatite is silanized and contains from 1 to 49 wt % of silicon, based on a total weight of the silanized hydroxyapatite, and at least 97% of the dentin tubules are occluded.

2: The method of claim 1, further comprising applying the toothpaste into a mouth tray wherein the dentin tubules are contacted with the toothpaste present in the mouth tray.

3: The method of claim 1, wherein the toothpaste comprises from 10 to 30 wt % of theobromine, based on a total weight of the theobromine and the bioactive glass and hydroxyapatite.

4: The method of claim 1, wherein the toothpaste comprises the bioactive glass and from 5 to 95 wt % of hydroxyapatite, based on a weight of the hydroxyapatite and the bioactive glass.

5: The method of claim 1, wherein the toothpaste further comprises from 10 to 50 wt % of fluorapatite, based on a weight of the fluorapatite and hydroxyapatite.

6: The method of claim 1, wherein at least 99% of the dentin tubules are occluded.

7: The method of claim 1, wherein the bioactive glass comprises from 40 to 80 wt % of silica and from 20 to 50 wt % of calcium oxide, based on a weight of the bioactive glass.

8: The method of claim 7, wherein the bioactive glass is at least one selected from the group consisting of 45S5, S53P4, and 13-93.

9: The method of claim 8, wherein the bioactive glass is a mixture of 45S5 and from 10 to 50 wt % of S53P4, based on a weight of 45S5 and S53P4.

10: A method for remineralizing teeth, the method comprising:
contacting the teeth with a toothpaste for 1-5 minutes, wherein the toothpaste comprises a bioactive glass, at least one of γ-poiyglutamic acid and γ-polyglutamate, and from 10 to 50 wt % of a casein phosphopeptide, based on a weight of the bioactive glass and casein phosphopeptide, and a pH of the toothpaste when mixed with saliva is more than 7.3 and less than 8.

11: The method of claim 10, further comprising applying the toothpaste into a mouth tray wherein the teeth are contacted with the toothpaste present in the mouth tray.

12: The method of claim 10, wherein the bioactive glass comprises from 40 to 80 wt % of silica and from 20 to 50 wt % of calcium oxide, based on a weight of the bioactive glass.

13: The method of claim 12, wherein the bioactive glass is at least one selected from the group consisting of 45S5, S53P4, and 13-93.

14: The method of claim 13, wherein the bioactive glass is a mixture of 45S5 and from 1.0 to 50 wt % of S53P4, based on a weight of 45S5 and S53P4.

15: The method of claim 12, wherein the toothpaste further comprises a fluoride source, and at least one of tri-calcium phosphate and amorphous calcium phosphate.

16: The method of claim 15, wherein the fluoride source is at least one selected from the group consisting of sodium fluoride, stannous fluoride, olaflur, and sodium monofluorophosphate.

17: The method of claim 15, wherein the toothpaste comprises from 10 to 50 wt % of the tri-calcium phosphate, based on a weight of the fluoride source and tri-calcium phosphate.

18: The method of claim 15, wherein the toothpaste comprises the tri-calcium phosphate and from 10 to 50 wt % of the amorphous calcium phosphate, based on a weight of the tri-calcium phosphate and amorphous calcium phosphate.

19: The method of claim 1, wherein the toothpaste comprises at least one of theophylline and paraxanthine.

20: The method of claim 10, wherein the toothpaste comprises at least one γ-polyglutamate selected from the group consisting of sodium γ-polyglutamate, potassium γ-polyglutamate, calcium γ-polyglutamate, magnesium γ-polyglutamate, and zinc γ-polyglutamate.
